(19) United States
(12) Patent Application Publication
(10) Pub. No.: US 2026/0253736 A1
(43) Pub. Date: Aug. 27, 2026

(54) ANOMALOUS PATIENT RECOVERY DETECTION

(71) Applicant: KONINKLIJKE PHILIPS N.V., EINDHOVEN (NL)

(72) Inventors: LIEKE GERTRUDA ELISABETH COX, EINDHOVEN (NL); MEIKE VAN DEN EIJNDEN, EINDHOVEN (NL); WILHELMUS FRANCISCUS JOHANNES VERHAEGH, Heusden gem. Asten (NL); WARNER RUDOLPH THEOPHILE TEN KATE, WAALRE (NL); GABRIELE PAPINI, EINDHOVEN (NL)

(21) Appl. No.: 18/861,335

(22) PCT Filed: Apr. 19, 2023
(86) PCT No.: PCT/EP2023/060054

§ 371 Date: Oct. 29, 2024

Related U.S. Application Data
(60) Provisional application No. 63/336,479, filed on Apr. 29, 2022.

(51) Int. Cl. G16H 50/30

(52) U.S. Cl. CPC G16H 50/30

(57) ABSTRACT

A system includes a memory that stores instructions; and a processor that executes the instructions. When executed by the processor, the instructions cause the system to repeatedly obtain a plurality of types of data derived from a recovering patient; apply the plurality of types of data to a trained model; repeatedly generate a recovery score predicting recovery of the recovering patient; and output the recovery score.

### BACKGROUND

After certain medical procedures such as surgeries, events such as cardiac arrest, or onset of diseases such as Covid-19, patients may recover along different pathways. Recovery is a complex process and no universally accepted definition exists. Recovery may be defined as the decrease in illness of a patient to or past the point where a health status before the illness is regained, and therefore, is a period in which the patient's health is changing, and most of the time improving. Recovery may also be defined as the ability of the patient to return to activities of daily life. A metric that is commonly used in hospitals to describe recovery is length of stay (LOS), but length of stay only reflects a single point in time, and not the path towards discharge. Furthermore, discharge decisions are often highly subjective in nature and are influenced by factors external to patient health, such as ability to return to home and hospital logistics. The end of the recovery period may be defined as the moment the patient is on the same level in health as before the surgery/disease or even better, or when patient health reaches a plateau/is no longer improving. The end of the recovery period may also be defined as when the patient reaches a sufficient level in health status together with sufficient trust in further recovery such that the patient may be discharged.

Recovery pathways may vary such as by being fast or slow, and/or with regularity in the development or with fluctuations. Recovery pathways may vary for reasons such as co-morbidities, demographic characteristics such as age, compliance with administered treatments and more. Insights as to a patient's recovery can support clinicians in assessing patient stability, discharge readiness and deterioration. To measure recovery over time, functional assessments or questionnaires may be used. However, functional assessments are time-consuming and may only cover a brief period of time, whereas questionnaires lead to subjective assessments of recovery at discrete points in time. To support clinicians in recovery-based decisions, such as assessing patient stability, discharge readiness and deterioration, early warning score (EWS) systems have been developed. Early warning systems utilize multiple parameters which are measured with spot checks during nursing rounds.

Functional assessments, questionnaires and early warning scores have limitations including the absence of standardization, subjectiveness, low amounts of data at discrete points in time, and high workload. Reliance only on spot checks leaves an absence of relevant data from times outside of the spot checks. Furthermore, spot checks highly rely on nurse time and are, therefore, time-consuming and labor-intensive. Moreover, the current high-level, generalized procedures for discharging patients may lead to uniform protocols that direct the discharge of every patient along a similar, pre-defined route. Little feedback is incorporated to control the pathway, and this can cause patients to stay in the hospital longer than needed, but also may cause delays in detecting anomalies in patient recovery.

### SUMMARY

According to an aspect of the present disclosure, a system includes a memory that stores instructions; and a processor that executes the instructions. When executed by the processor, the instructions cause the system to repeatedly obtain a plurality of types of data derived from a recovering patient; apply the plurality of types of data to a trained model; repeatedly generate a recovery score predicting recovery of the recovering patient; and output the recovery score.

According to another aspect of the present disclosure, a central system includes a memory that stores instructions; and a processor that executes the instructions. A method performed by the central system includes repeatedly obtaining a plurality of types of data derived from a recovering patient; applying, by the processor that executes instructions from the memory, the plurality of types of data to a trained model; repeatedly generating a recovery score predicting recovery of the recovering patient; and outputting the recovery score.

According to another aspect of the present disclosure, a tangible non-transitory computer readable storage medium stores a computer program. The computer program, when executed by a processor, causes a system to: repeatedly obtain a plurality of types of data derived from a recovering patient; apply the plurality of types of data to a trained model; repeatedly generate a recovery score predicting recovery of the recovering patient; and output the recovery score.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1 illustrates a system for anomalous patient recovery detection, in accordance with a representative embodiment.

FIG. 2 illustrates another system for anomalous patient recovery detection, in accordance with a representative embodiment.

FIG. 3 illustrates a method for anomalous patient recovery detection, in accordance with a representative embodiment.

FIG. 4 illustrates another method for anomalous patient recovery detection, in accordance with a representative embodiment.

FIG. 5A, FIG. 5B, FIG. 5C, FIG. 5D, FIG. 5E and FIG. 5F illustrate recovery profiles for recovering patients, in accordance with a representative embodiment.

FIG. 6 illustrates correlations between recovery scores and features per day, in accordance with a representative embodiment.

FIG. 7A illustrates predicted recovery versus expected recovery, in accordance with a representative embodiment.

FIG. 7B illustrates recovery scores over time, in accordance with a representative embodiment.

FIG. 8A, FIG. 8B and FIG. 8C illustrate individual predicted profiles compared to average profiles, in accordance with a representative embodiment.

FIG. 9 illustrates predicted average recovery profiles, in accordance with a representative embodiment.

FIG. 10 illustrates a computer system, on which a method for anomalous patient recovery detection is implemented, in accordance with another representative embodiment.

### DETAILED DESCRIPTION

In the following detailed description, for the purposes of explanation and not limitation, representative embodiments disclosing specific details are set forth in order to provide a thorough understanding of an embodiment according to the present teachings. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only and is not intended to be limiting. The defined terms are in addition to the technical and scientific meanings of the defined terms as commonly understood and accepted in the technical field of the present teachings.

It will be understood that, although the terms first, second, third etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

The terminology used herein is for purposes of describing particular embodiments only and is not intended to be limiting. As used in the specification and appended claims, the singular forms of terms ‘a,’ ‘an’ and ‘the’ are intended to include both singular and plural forms, unless the context clearly dictates otherwise. Additionally, the terms “comprises”, and/or “comprising,” and/or similar terms when used in this specification, specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to,” “coupled to,” or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components. The present disclosure, through one or more of its various aspects, embodiments and/or specific features or sub-components, is thus intended to bring out one or more of the advantages as specifically noted below. For purposes of explanation and not limitation, example embodiments disclosing specific details are set forth in order to provide a thorough understanding of an embodiment according to the present teachings. However, other embodiments consistent with the present disclosure that depart from specific details disclosed herein remain within the scope of the appended claims.

As described herein, patient recovery over time may be objectively tracked such that anomalous recovery may be identified to help clinicians make informed recovery-based decisions.

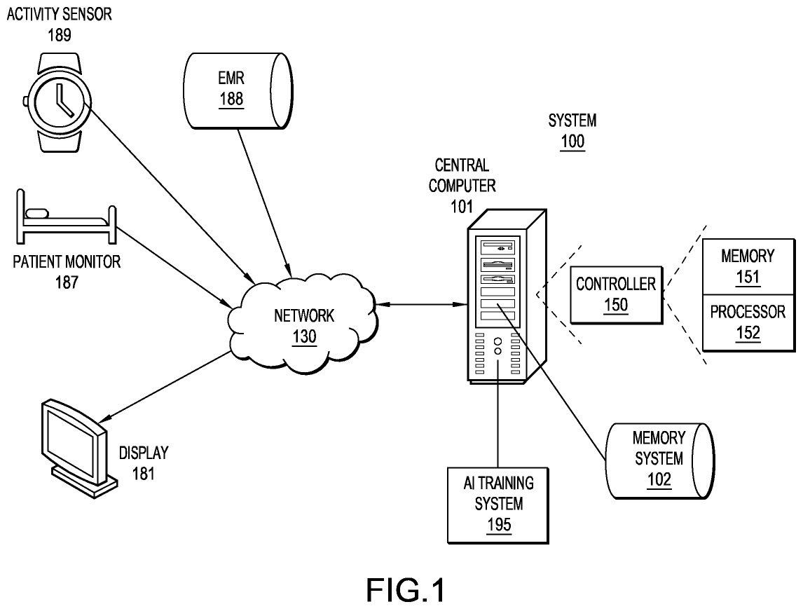

FIG. 1 illustrates a system for anomalous patient recovery detection, in accordance with a representative embodiment.

The system 100 in FIG. 1 is a system for anomalous patient recovery detection and includes components that may be provided together or that may be distributed. The system 100 includes a central computer 101, a memory system 102, a network 130, a display 181, a patient monitor 187, an EMR 188, an activity sensor 189, and an AI training system 195.

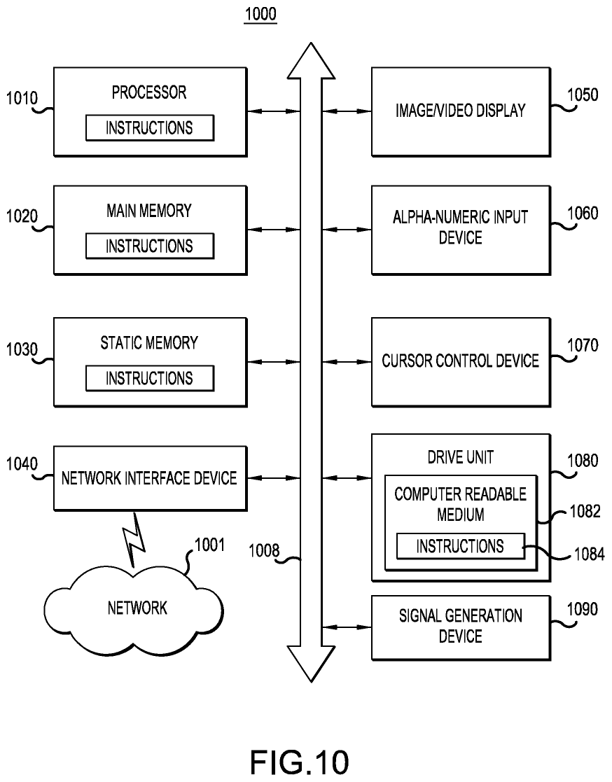

The central computer 101 may comprise an individual computer or may alternatively represent a central system comprising multiple individual computers. For example, the central computer 101 may represent a system comprising one or more servers provided on behalf of an organization that provides an anomalous patient recovery detection service. The central computer 101 may represent a cloud-based system with multiple serves provided in a data center. A computer that can be used to implement the central computer 101 is depicted in FIG. 10, though a central computer 101 may include more or fewer elements than depicted in FIG. 1 or FIG. 10.

The central computer 101 includes at least a controller 150, and the controller 150 includes at least a memory 151 that stores instructions and a processor that executes the instructions. In embodiments in which multiple computers are used to provide functionality attributed herein to the central computer 101, each of the multiple computers may include a controller such as the controller 150 and, in turn, a memory such as the memory 151 and a processor such as the processor 152. The controller 150 executes instructions to implement some or all features attributed to the central computer 101 herein. The controller 150 may perform some or all of the operations described herein directly. For example, the controller 150 may directly control operations such as logical operations performed by the processor 152 executing instructions from the memory 151 based on data derived from a recovering patient via the patient monitor 187 and the activity sensor 189. The processes implemented by the controller 150 when the processor 152 executes instructions from the memory 151 may also include steps not directly performed by the controller 150.

The memory system 102 stores data for the central computer 101 and may be provided with the central computer 101. For example, the memory system 102 may store a trained prediction model used to generate recovery scores predicting recovery of recovering patients, as well as types of baselines which may be established on a dynamic basis to compare with recovery scores. The central computer 101 may retrieve and apply the trained prediction model to generate recovery scores, and may dynamically generate or select and retrieve a baseline to compare with each recovery score.

The network 130 may comprise a broadband wide area network (WAN) such as the internet, and may include wired and wireless communications equipment by which data may be transmitted directly and indirectly between patients and the central computer 101.

The display 181 may be a monitor such as a computer monitor, a display on a mobile device, an augmented reality display, a television, an electronic whiteboard, or another screen configured to display electronic imagery. The display 181 is representative of a medical monitor that displays recovery scores received from the central computer 101. The display 181 may be closely located in proximity to the patient monitor 187 and/or the activity sensor 189 around a patient. In some embodiments, the display 181 may be integrated with the patient monitor 187 and/or the activity sensor 189, so that recovery scores and alarms from the central computer 101 are provided on a user interface of the display 181 together with types of data obtained from the patient monitor 187 and the activity sensor 189. The display 181 may also be integrated with a computer or another type of electronic device, such as in an office apart from a patient room in a hospital. The display 181 may be or include a user interface of a patient monitor such as the patient monitor 187, so that recovery scores from the central computer 101 may be displayed along with measurements of blood pressure, SpO2, ECG etc.

The patient monitor 187 and the activity sensor 189 are representative of electronic equipment used to obtain a plurality of types of data from recovering patients. The plurality of types of data may include data that are derived from vital signs of the recovering patients, and/or may include data that are derived from activity metrics for the recovering patients. The plurality of types of data may include data specifically identified as being most relevant to a trained prediction model executed by the central computer 101, though other types of data may also be obtained and provided to the central computer 101. Examples of the patient monitor 187 includes an SpO2 monitor, a blood pressure monitor, a heart rate monitor, a respiration monitor, and other types of medical monitors that monitor physiology of patients. Examples of the activity sensor 189 include wearable fitness monitors such as watches that monitor physiology of users, and other types of electronic devices including smartphones with fitness applications installed thereon. Input data for the central computer 101 may be collected for patients using wearables with PPG and acceleration sensors. Data from wearable sensors including ECG, capnography, EMG, barometers, temperature sensors, gyroscopes, galvanic skin response, etc. may also be used. The activity sensor 189 may also be representative of sensors that are not specifically measuring activity, such as measurements when a patient is resting or unconscious. Additionally, more than one patient monitor may be represented by the patient monitor 187, and more than one activity sensor or other type of sensor may be represented by the activity sensor 189.

The EMR 188 is representative of an electronic medical records system for a health organization such as a hospital or hospital system. The EMR 188 may store patient records for the patients being monitored by the patient monitor 187 and using the activity sensor 189. The electronic medical records stored in the EMR 188 may include notes and measurements taken by medical personnel such as doctors, nurses and technicians. The patient records may include vital signs and activity metrics captured by the patient monitor 187 and the activity sensor 189 and automatically provided over a local network for storage in the EMR 188. The patient records may also include vital signs and activity metrics captured by medical personnel and entered into electronic user interfaces and then provided over a local network for storage in the EMR 188. Data from the EMR 188 may include data from spot check measurements including blood pressure or biomarker measurements such as from blood samples, weight, cardiac output, or questionnaires, and other types of EMR data such as medication data.

As set forth above, the display 181, the patient monitor 187, the EMR 188 and the activity sensor 189 may be co-located at a medical facility such as a hospital. In some embodiments, however, patients may be monitored for anomalous patient recovery detection even after they are released from such medical facilities, so that the patient monitor 187 and/or the activity sensor 189 are not necessarily co-located with the display 181 and the EMR 188.

In the embodiment of FIG. 1, a medical facility may include multiple patients each provided with multiple patient monitors such as the patient monitor 187 and with multiple activity sensors such as the activity sensor 189. For example, multiple patients at a single medical facility may simultaneously obtain the services of the central computer 101 over the network 130. Each such patient may also provide vital signs and activity metrics as well as data derived from the vital signs and activity metrics from the EMR 188 and from instances of the patient monitor 187 and the activity sensor 189 to the central computer 101 over the network 130.

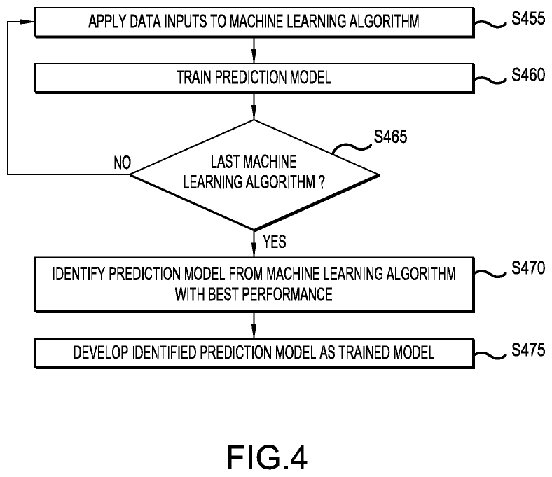

The AI training system 195 is representative of a system used to train artificial intelligence such as a trained model which is used to repeatedly generate recovery scores for patients. The AI training system 195 may also be used to develop other types of prediction models and algorithms, and is not limited to training only artificial intelligence. The AI training system 195 may apply numerous types of data inputs to machine learning algorithms to identify which of the data inputs provide the best correlations with expected results, and which of the machine learning algorithms can be used to train a prediction model with the best performance compared to others of the machine learning algorithms. An example process for developing a prediction model as a trained model using machine learning algorithms is shown in and described with respect to the method of FIG. 4. The AI training system 195 may be provided by an entity that provides the central computer 101, such as by a health care provider, or may be provided independently as a third-party service for multiple different entities such as multiple different health care providers. As an example, the AI training system 195 may include one or more computers, including one or more servers, to develop the trained model applied by the central computer 101 to generate recovery scores predicting recovery of recovering patients.

As set forth above, the central computer 101 may be representative of a system that includes a memory that stores instructions and a processor that executes the instructions. Such a system may repeatedly obtain a plurality of types of data derived from a recovering patient, and apply the plurality of types of data to a trained model each time the plurality of types of data are obtained. For example, the central computer 101 may apply the trained model to the plurality of types of data obtained from a patient periodically, or at least repeatedly when sufficient data is made available. By applying the trained model to the plurality of types of data, such a system may repeatedly generate a recovery score predicting recovery of the recovering patient, and may output the recovery score. In FIG. 1, the system which includes the central computer 101 is centralized and outputs recovery scores for a plurality of recovering patients.

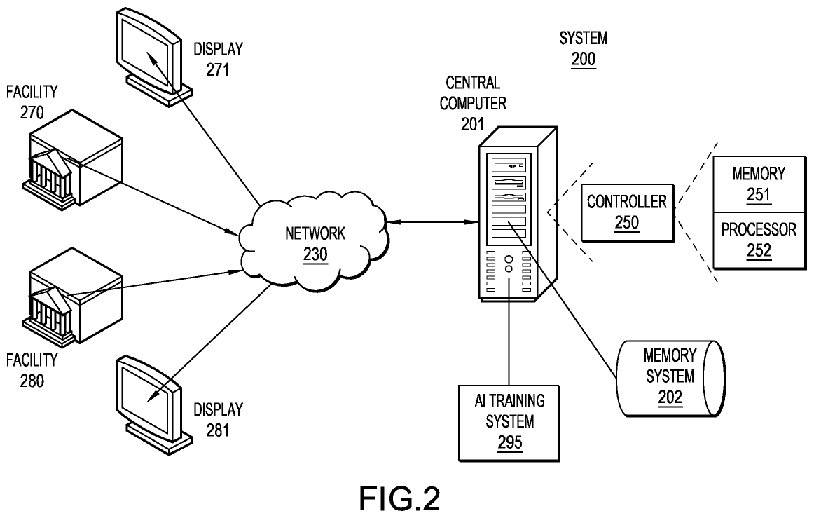

FIG. 2 illustrates another system for anomalous patient recovery detection, in accordance with a representative embodiment.

The system 200 in FIG. 1 is a system for anomalous patient recovery detection and includes components that may be provided together or that may be distributed. The system 200 includes a central computer 201, a memory system 202, a network 230, a facility 270, a display 271, a facility 280, a display 281, and an AI training system 295. Each of the facility 270 and the facility 280 in FIG. 2 may correspond to instances of a facility of FIG. 1 which includes the display 181, the EMR 188, the patient monitor 187 and the activity sensor 189. The display 271 and the display 281 in FIG. 2 may each correspond to instances of the display 181 in FIG. 1. The AI training system 295 in FIG. 2 may correspond to the AI training system 195 in FIG. 1.

In the embodiment of FIG. 2, multiple medical facilities may each include multiple patients each provided with multiple patient monitors and with multiple activity sensors. For example, multiple patients at multiple different medical facilities may simultaneously obtain the services of the central computer 201 over the network 230. Additionally, patients at each of the facility 270 and the facility 280 may send a plurality of types of data to the central computer 201, and the central computer 201 may apply the plurality of types of data to a trained model each time the plurality of types of data are obtained.

As set forth above, the central computer 201 may be representative of a system that includes a memory that stores instructions and a processor that executes the instructions. Such a system may repeatedly obtain a plurality of types of data derived from a recovering patient, and apply the plurality of types of data to a trained model each time the plurality of types of data are obtained. For example, the central computer 201 may apply the trained model to the plurality of types of data obtained from a patient periodically, or at least repeatedly when sufficient data is made available. By applying the trained model to the plurality of types of data, such a system may repeatedly generate a recovery score predicting recovery of each of the recovering patients, and may output the recovery scores. In FIG. 2, the system which includes the central computer 201 is centralized and outputs recovery scores for a plurality of recovering patients. In FIG. 2, the system which includes the central computer 201 also generates and outputs recovery scores for recovering patients of a plurality of medical facilities.

Using the system 100 and the system 200, data and metadata for a patient may be compared with data and metadata from other patients with similar conditions and with other conditions, from the same hospital and from other hospitals. Anomalies in the patient or patient group may be detected, so that anomalous patient recoveries may be individually detected and anomalous patient recovery trends may be detected for groups of patients such as at specific hospitals or other care facilities. The data provided to the central computer 101 and the central computer 201 may include health state information of patients over time. The collected health state information may include vital signs such as heart rate, respiration rate, blood pressure, etc. The collected health state information may also include activity metrics such as steps, activity type, posture, walking speed and distance, activity level, activity counts, etc. The data provided to the central computer 101 and the central computer 201 may also include demographic data such as age, height, weight and gender, and medical record information such as disease classification, surgery type, date of surgery, medications, and lab tests. Data collection may be performed via wearables, patient monitors, EMR systems, spot checks and more. Combinations of collected data may be converted into sets of trends per patient. The sets of trends per patient may be compared to reference sets of trends from similar patients, such as patients who are subjected to the same surgery, have the condition, are the same age. Anomalous recovery may be detected by the central computer 101 and the central computer 201, and health care professionals may be guided in recovery-based decisions.

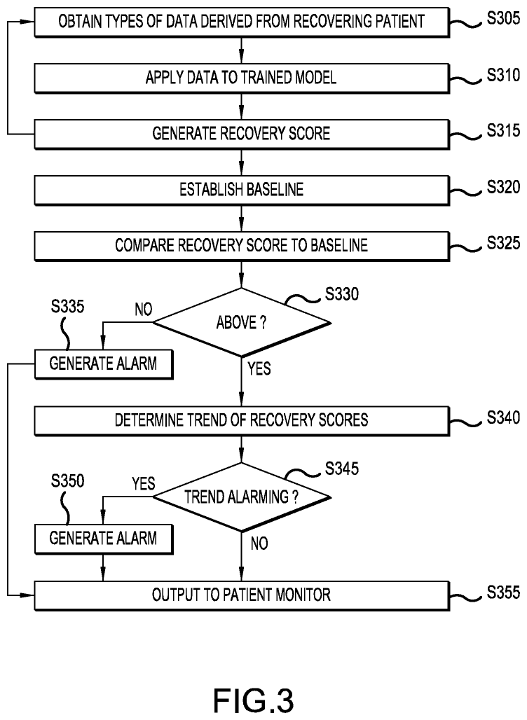

FIG. 3 illustrates a method for anomalous patient recovery detection, in accordance with a representative embodiment.

The method of FIG. 3 may be performed by the central computer 101 in FIG. 1 or the central computer 201 in FIG. 2. The method of FIG. 3 starts at S305 by obtaining types of data derived from a recovering patient. Data of multiple types may be continuously or semi-continuously collected in an automated fashion for processing through a trained model implemented at the central computer 101 in FIG. 1 or the central computer 201 in FIG. 2.

For example, the types of data derived from a recovering patient may include measurements of a patient recovering from a treatment such as a cardiac intervention or an event such as cardiac infarction. The types of data may include data that are derived from measurements of vital signs of the patient, and/or may include data that are derived from activity metrics for the patient. Other forms of data may also be relevant to the trained model described herein. For example, types of data obtained at S305 may also include metrics such as biomarkers, demographic information, questionnaire answers such as for questions as to pain levels, and more.

At S310, the data obtained at S305 is applied to a trained model. The trained model may be a prediction model trained by the AI training system 195 or the AI training system 295. For example, the trained model may comprise an extreme gradient boosting-based model. Other types of trained models may also be used as the trained model implemented by the central computer 101 or the central computer 201. For example, linear regression with Lasso, k-nearest neighbor, tree-based methods such as decision tree or random forest, and support vector regression may be used to develop the trained model implemented by the central computer 101 or the central computer 201. The trained model may apply a data set to determine whether the patient is recovering on an objective basis. As explained elsewhere herein, the trained model may take a subset of data derived from measurements of vital signs and derived from activity metrics as the trained model may be trained to recognize a particular subset of data that is most relevant to determining an objective health state

At S315, a recovery score is generated for the patient based on applying the trained model to the data at S310. For example, a recovery score may comprise a value between 1.0 and 2.0, with 1.8 serving as the threshold for whether a patient is ready to be discharged. Recovery may be predefined such that the recovery score may be an objective measure relative to the definition. For example, the end of the recovery period may be defined as the moment the patient is on the same level in health as before the surgery/disease or even better, or when patient health reaches a plateau and is no longer improving. The end of the recovery period may also be defined as when the patient reaches a sufficient level in health status together with sufficient trust in further recovery such that the patient may be discharged.

As an alternative to predicting a recovery score at S315, discharge readiness may be predicted. Discharge readiness may be defined, for example, using a recovery score of 1 or below as 0% discharge ready, and 1.8 and above as 100% discharge ready. Also, similar predictions may be used to predict the discharge date, such as by extrapolating the recovery scores to 1.8, or by comparing to different length of stay groups such as those most likely to fit the 5-7 day length of stay group.

The process from S305 to S315 is repeated. For example, each time an appropriate set of data is obtained at S305, the set of data may be applied to the trained model at S310 so that a recovery score is generated at S315. The process from S305 to S315 may be performed periodically rather than continuously, such as every 5 minutes, every hour, every 6 hours, every 12 hours or every 24 hours. Additionally, the process from S305 to S315 may be performed intermittently, such as once the appropriate set of data is obtained at S305 when the appropriate set of data is not otherwise obtained on a continuous basis.

At S320, a baseline is established for the recovery score generated at S315. The baseline is not necessarily fixed, and instead may be selected from a set of baseline types stored in the memory system 102 or the memory system 202. A set of baseline classes may be generated and may vary based on any number of criteria including, but not limited to, type of treatment or event, condition of the patient prior to the treatment or event, recovery scores of previous similar patients who recovered from the same or similar treatment or event, demographic characteristics of the patient such as age and/or weight, and time since the treatment or event. For example, a baseline used for comparison to evaluate whether the patient is recovering successfully may increase over time based fundamentally on expectations that patients improve over time if they are recovering. Therefore, establishing a baseline at S320 may include dynamically generating a baseline based on a set of variable values input to an equation, or may include selecting a baseline class from a predetermined set of baseline classes.

At S325, the recovery score generated at S315 is compared to the baseline established at S320. For example, the recovery score and a comparable value of a baseline curve may be directly compared. The comparable value of a baseline curve may reflect the amount of time which has passed since the treatment or event, insofar as baseline curves increase over time.

At S330, a determination is made as to whether the recovery score generated at S315 is above the baseline established at S320. The determination at S330 may be a simple yes or no, or may comprise a relative value that reflects the distance of the recovery score from the baseline, particularly when the recovery score is below the baseline. For example, the baseline may be a class or set of values. The determination at S330 may also be whether the recovery score is within a baseline range, or a member of a baseline family or class or set, and is not specifically required to be above a baseline. The comparison may also reflect a variance that is tolerable in a time series of measured values, such that tolerance may be provided within a defined or definable range for recovery scores so that the recover scores may be larger or smaller than the actual threshold.

If the recovery score is not higher than the baseline (S330=No), at S335 an alarm is generated. The nature of the alarm may vary based on the relative distance of the recovery score from the baseline. For example, a recovery score that is far below the baseline may result in a notification to an attending physician, whereas a recovery score that is close to the baseline but still below the baseline may result in a notification to the display 181 in the patient's room at a hospital. An alarm may be persistently displayed on the display 181, such as until the recovery score is above the baseline at a subsequent comparison or until the alarm is turned off by an authorized user. In some embodiments, a patient that is recovering better than expected may also result in an alarm, such as to alert a doctor or nurse to check on the recovering patient to be sure the recovering patient is recovering better than expected, or to enable timely discharge planning.

If the recovery score is higher than the baseline (S330=Yes), at S340 a trend of recovery scores is determined. The trend may comprise a profile that includes the recovery score generated at S315 and previous recovery scores generated for the same patient. For example, if the trend is not improving or is specifically worsening, it may be indicative of a problem. The trend may comprise recovery scores for each of two or more days. Additionally, the trend may comprise a comparison with the relevant baseline established at S320, such as when the same baseline is used for a patient throughout the patient's recovery.

In some embodiments, the trend of recovery scores may be determined at S340 even when the recovery score is not higher than the baseline. For example, the trend of recovery scores may always be investigated, independent of whether a recovery score is above a baseline at any particular point in time. Trends of recovery scores may be investigated with both high recovery scores and low recovery scores. Decreasing trends or prematurely plateauing trends may be identified and result alarms in the manner described herein.

At S345, a determination is made whether the trend is alarming. For example, a trend may be deemed alarming if the trend is worsening or simply not improving. The determination at S345 may reflect consideration of only the two most recent recovery scores, or may reflect consideration of more than two of the most recent recovery scores, such as when a recovery score fails to improve for two or more days.

If the trend is alarming at S345 (S345=Yes), an alarm is generated at S350. The trend determined at S340 is a trend of recovery scores generated, for example, each day. However, in other embodiments, an estimation of the future recovery score values may also or alternatively be determined so that the future trend itself may be predicted. The trend prediction may be based on the data collected in the previous days and, additionally, the recovery trends of other patients. The predicted trend may also be associated with a confidence interval. For example, the upper boundary of a confidence interval may be calculated using the trends from fast recovery patients, while the lower boundary of the confidence interval may be calculated using the trends from slow recovery patients.

If the trend is not alarming at S345 (S345=No), or otherwise after the alarm is generated at S335 or the alarm is generated at S350, the recovery score and any alarm are output to a patient monitor at S355. The relevant baseline may also be output at S355, so that the relevance of the recovery score to the baseline may be visually inspected. For example, recovery scores, alarms and baselines may be output from the central computer 101 to the display 181 in FIG. 1 or from the central computer 101 to the display 271 or the display 281 in FIG. 2. Additionally, the recovery scores and/or alarms are not necessarily output to a medical monitor, or only to a medical monitor. For example, recovery scores and/or alarms may be output to mobile devices such as (hospital-issued) smartphones, central monitoring stations such as nurse stations at a hospital, other types of networked communications devices.

The output at S355 may be to an output device such as a patient monitor, such as at a medical facility. The output at S355 may also or alternatively be to a central monitor, to an application on a mobile device, to the mobile device directly such as by a text message, or in another form of electronic transmission.

The output at S355 may result in a visualization of profiles of individual patients overlaid on a short length of stay group or a long length of stay group, and this may support health care providers in identifying patients with anomalous recoveries. However, health care providers may not have time to analyze such plots for all patients every day. Thus, alerts may be provided as the output at S355, based on thresholds or trends. For example, group profile boundaries may be set, so that alarms are sent to health care providers when recovery scores are outside of the group profile boundaries. In the example use case, if a patient is outside the lower boundary of the confidence interval for the short length of stay group, a flag may be output to indicate the patient is likely to have a long length of stay. In general, if a patient is deviating outside the band of a group, the deviation may be provided to the care giver. If the patient is also outside the lower boundary of the confidence interval of long length of stay, a warning may be provided to have a closer look at the recovery scores and the values of the model features (activity, vital signs etc) due to anomalously slow recovery. Also, the confidence of such flags may be reported, such as when the flags may be influenced by data completeness/incompleteness and quality. For example, fewer than all features may be calculated when data is missing, and this may affect model accuracy. Additionally, the number of days a patient is below or above a certain threshold and the distance to such thresholds may also be provided as output to a health care provider.

Another metric which may be observed and output at S355 is the variation of the patient's recovery curve. For example, a reference curve may be determined as the smoothed version of the mean curve in a patient group's band. Then, the deviation of the actual patient's data to this reference curve may be computed and the absolute value of all values may be summed and divided by the number of points included. Alternatively, a smoothed curve may be fit to the patient's data and the deviation to that fit may be computed. The deviation may be a sum of absolute difference values or a sum of squared differences, both divided by the number of points and where the square root is computed of the sum of squares. Other methods to obtain a scale of variance are known in the art, and may be used.

Aside from thresholds, trends in predicted recovery scores may also be used to decide on alerting health care providers. For example, a decline for multiple days/predictions may warrant an alert, even if the patient is not yet below a predefined threshold. Furthermore, instead of differentiating between groups with short and long length of stay, differentiations may be provided between groups with and without complication, or with and without long-term disabilities, or diverse levels of long-term independency etc. Differentiations are also not necessarily between two groups. For example, differentiations may be provided between short length of stay, medium length of stay, and long length of stay. Furthermore, different subgroups may be created. For example. a 90 year old patient who undergoes a particular surgery type might recover more slowly compared to a 50 year old patient who undergoes the same surgery type, so patients for this surgery type may be compared to the overall group and also to an age-matched subgroup. Differentiations may also be used to contextualize alerts to the health care providers. For example, an alert to a health care provider may indicate “Patient X is recovering slow compared to average after surgery Y (predicted length of stay of 11-13 days), but within the expected profile for his age group (80+), with an average length of stay of 13 days.”

As a result of the method of FIG. 3, a reliable and objective representation of patient recovery may be provided with a relatively-low workload compared to using only functional assessments, questionnaires and/or early warning scores. Moreover, the method of FIG. 3 is not limited in applicability to recovery after surgery. For example, recovery after disease onset of diseases such as Covid-19 may also be predicted using methods similar or the same as the method shown in FIG. 3, to help identify patients with slow recovery early on, even when they are recovering outside hospital, or after an event such as stroke or cardiac arrest.

FIG. 4 illustrates a method for anomalous patient recovery detection, in accordance with a representative embodiment.

The method of FIG. 4 starts at S455 by applying data inputs to a machine learning algorithm. The data inputs may include measurements of physiological characteristics obtained from patients, and data derived from such measurements. The data inputs may also include activity metrics obtained from patients, and data derived from such activity metrics. The machine learning algorithm may be one of multiple machine learning algorithms being tested to see which performs best in terms of developing an optimal trained model for use in the anomalous patient recovery detection described herein.

At S460, a prediction model is trained based on applying the data inputs to the machine learning algorithm at S455. The prediction model may be trained by repeatedly inputting a variety of types of data to optimize decisions by nodes of the prediction model using ground truth data. The ground truth used in the training at S460 may comprise ground truth recovery scores, though in some embodiments alternative ground truths may comprise proxies for recovery scores such as functional assessments or questionnaire scores.

If ground truth recovery scores are known for each patient, at different moments in time, such ground truth recovery scores may be used to build and train a model using machine learning techniques to predict recovery scores based on the input parameters such as the vital sign and activity metrics. To obtain reference recovery scores to train a model, a continuous recovery reference profile is engineered for the first set of embodiments. The continuous recovery reference profile is engineered based on events such as discharge, complications, readmissions,, and based also on literature and intuition. The functional recovery profiles follow an exponential trend. That is, large improvements in recovery are made in the first days, and after this, the profiles rise more slowly until reaching a plateau. Patients with distinct levels of recovery will plateau on different days. In addition, all patients may be given the same starting recovery score at the day of surgery, except for patients with a complication in the first days as these patients may be expected to go below the base level after surgery.

At S465, a determination is made whether the current machine learning algorithm is the last machine learning algorithm. For example, when the AI training system 195 or the AI training system 295 is training the trained model used by the central computer 101 or the central computer 201, the training may involve training multiple different machine learning algorithms of distinct types to determine which provides the best results.

If the current machine learning algorithm is not the last machine learning algorithm (S465=No), the process returns to S455 to start applying another (the next) machine learning algorithm to the data inputs.

If the current machine learning algorithm is the last machine learning algorithm (S465=Yes), at S470 the method of FIG. 4 includes identifying a prediction model from the machine learning algorithm with the best performance. To identify the prediction model from the machine learning algorithm with the best performance, the machine learning algorithms may be compared based on predictive performance and interpretability. Regarding the predictive performance, the machine learning algorithms may be judged on how well they fit to the reference recovery curve, utilizing the mean absolute percentage error. Next, the Spearman rank correlation coefficient may be calculated to ensure the trends of the predicted recovery profiles are correct. The Spearman rank correlation may reflect whether a rising trend exists and if a drop in recovery occurs when complications arise. Finally, the error at discharge may be investigated. The error at discharge may reflect how well the trained model predicted the same recovery score on the discharge day. Regarding the level of interpretability, more interpretable models may be preferred as this may lead to higher adoption in the clinic. Although the XGBoost model may be less interpretable than other algorithms, using only six features increases the ability of the XGBoost model to explain the outcomes.

Recovery detection may be defined as a classification problem, such as to predict if a patient would be in a long length of stay group or a short length of stay group. In case recovery detection is defined as a classification problem, predictive power may be expressed in metrics such as precision and recall. Alternatively, sensitivity and specificity may be used instead. These pairs of metrics may be summarized in scalar metrics such as F-score and area under curve (AUC). Additionally, in comparing designs it is common to use metrics such as correlation and kappa score. A method such as the Bland-Altman plot may be used instead or in addition.

At S475, the identified prediction model is developed as a trained model. The trained model may be provided to the central computer 101 or the central computer 201 for use as the trained model in the method of FIG. 3.

Model development based on the embodiment of FIG. 4 may also be aimed at alternatives to detecting anomalous recovery, such as predicting length of stay.

Although a recovery score per patient may be engineered for the trained model resulting from the method of FIG. 4, other ways to obtain reference scores for training a model may also be used, such as asking health care providers to provide recovery scores for a training set or by taking recovery proxies. Data from health care providers may be provided by multiple assessors to overcome limitations of subjectivity. Data from proxies may be taken from combinations of recovery proxies, such as functional assessments or questionnaire scores as ground truth, even though these have limitations. Also, it may be challenging to obtain enough good quality reference scores that represent and span the target patient population sufficiently. Recovery trajectories may be quite different for different conditions and interventions. In the event of planned surgeries, for example, data may be collected before the surgery, so that a baseline is available for each individual patient that can be used as input for the recovery score as well. In addition, an option to select the patients to calculate the different recovery trend groups may be implemented. The selection may be automatic based on the collected data or may be manual based on the health care provider input. Automated selection may be based, for example, on data quality, amount of data per patient, and patient characteristics, such as age, gender, BMI, etc.

Manual selection of data may include requesting the health care providers to include only patients with specific characteristics.

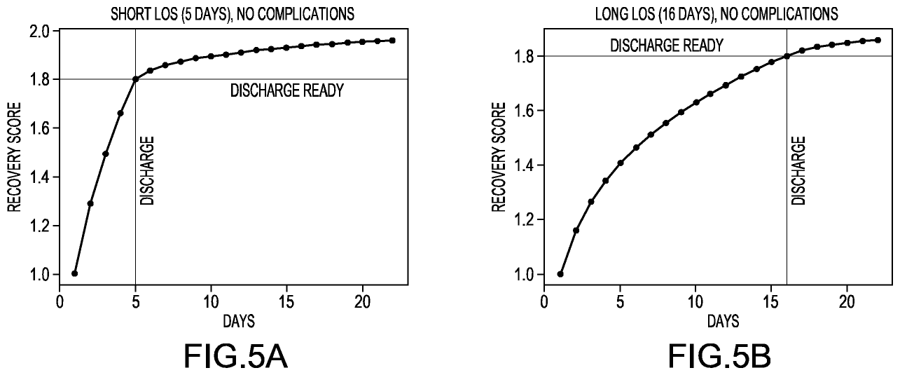

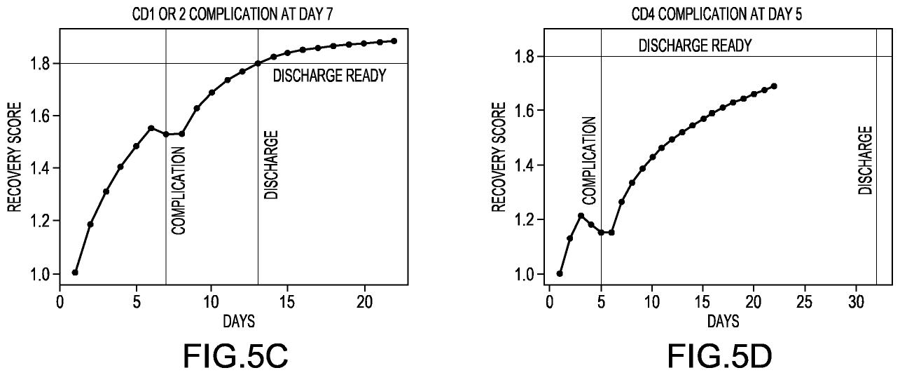

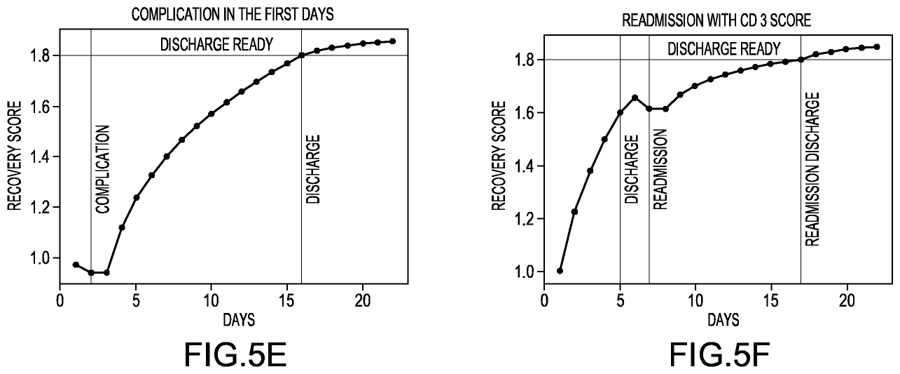

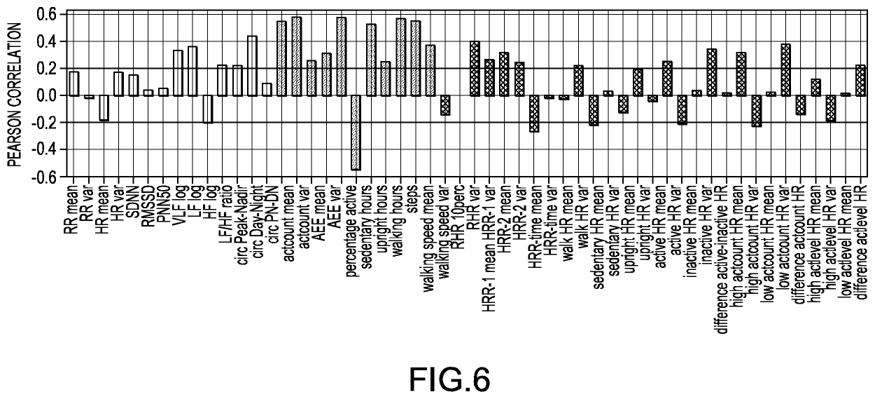

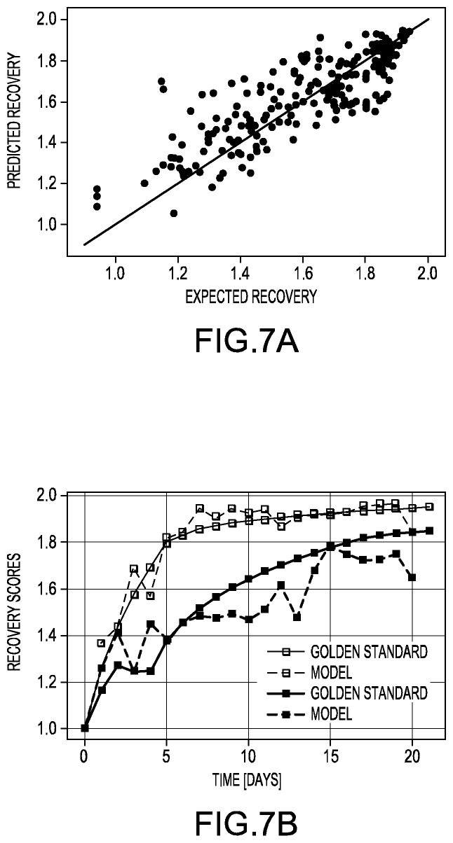

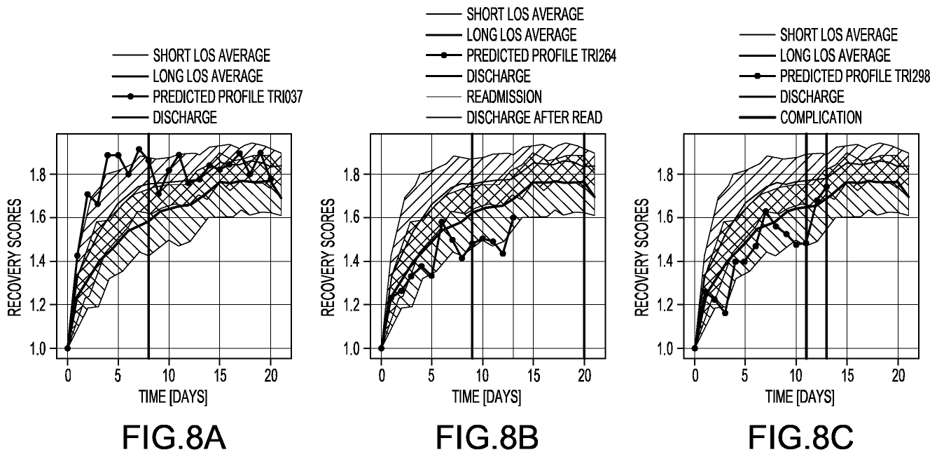

A first set of example embodiments is described below with respect to FIG. 5A, FIG. 5B, FIG. 5C, FIG. 5D, FIG. 5E, FIG. 5F, FIG. 6, FIG. 7A, FIG. 7B, FIG. 8A, FIG. 8B and FIG. 8C.

To explain the first set of example embodiments, health status parameters may be collected from a group of oncology patients undergoing abdominal surgery to remove tumors.

The health status parameters may be continuously collected from the day of the surgery until up to 3 weeks after, both in the hospital and at home. The health status parameters may include vital sign parameters such as heart rate, respiration rate, heart rate variability metrics, etc., derived from accelerometer, electrocardiogram (ECG), and photoplethysmography (PPG) sensor data, for example. The health status parameters may also include activity parameters such as number of steps, activity level, walking speed, and duration, posture, etc., derived from accelerometer sensor data. Combinations of the vital sign parameters and activity parameters may also be continuously collected. Examples of such combinations include recovery of heart rate after an activity, resting heart rate, heart rate during walking, and more.

FIG. 5A, FIG. 5B, FIG. 5C, FIG. 5D, FIG. 5E and FIG. 5F illustrate recovery profiles for recovering patients, in accordance with a representative embodiment.

The engineered recovery profiles in FIG. 5A, FIG. 5B, FIG. 5C, FIG. 5D, FIG. 5E and FIG. 5F are for six oncology surgery patients, showing the influence of discharge day, complications with different severities as expressed by Clavien-Dindo (CD) scores, and readmissions. As can be seen from FIG. 5A, FIG. 5B, FIG. 5C, FIG. 5D, FIG. 5E and FIG. 5F, the resulting recovery profiles are smooth, with disruptions only in case of complications or readmissions, whereas the actual profiles are likely to be more fluctuating. However, this does not need to be a problem for developing and training a model as in FIG. 4.

The recovery curves for the first set of embodiments are designed to increase from 1.0 to 2.0. On the day of discharge from the hospital, a score of 1.8 is assigned, assuming a recovery of 80%, so the patients are expected to recover further at home. Utilizing this discharge date of the patient, the reference recovery profiles differ for patients with short length of stay and long length of stay, as can be seen in FIG. 5A and FIG. 5B. If a complication occurs, a drop in recovery score is created, starting one or two days before the actual complication as there may be a lead time. The size of this drop is based on the severity of the complication, as indicated by the Clavien-Dindo classification on a scale from 1 to 5, where 5 represents death of the patient. A small drop occurs if a complication arises with a Clavien-Dindo classification of 1 or 2. If a complication arises with a higher Clavien-Dindo grade, a larger drop occurs. For example, a complication with Clavien-Dindo grade 4 requires the patient to go to the ICU, which causes a considerable drop in recovery. Readmissions are engineered the same way as complications, based on the Clavien-Dindo classification. However, differences are made in the recovery scores at discharge from the hospital. The discharge score for the first discharge is changed to 1.6, as the patients may be unlikely to be discharge-ready since readmission occurred. As a result, the discharge after the readmission may be valued at a recovery score of 1.8. FIG. 5F shows an example of a readmission with a Clavien-Dindo score of 3.

A machine learning approach may be used to predict recovery scores of oncology abdominal surgery patients. The aim may be to develop a trained model that takes the observed values and time series as input and outputs a recovery score or expectation. The input to the machine learning algorithm for the training phase may include features and the engineered reference profiles of the training set. As can be seen from FIG. 5A, FIG. 5B, FIG. 5C, FIG. 5D, FIG. 5E and FIG. 5F, the engineered references are continuous profiles and may be used to predict recovery scores at different timescales, but which are used in the first set of embodiments for daily recovery scores.

Therefore, per measured parameter, a value per day may be calculated to be used as an input feature. The measured parameters may include total amount of steps per day, mean heart rate, total energy expenditure, number of hours spent upright, etc. These types of data may comprise the types of data repeatedly obtained and applied to a trained model by the central computer 101 and the central computer 201.

FIG. 6 illustrates correlations between reference recovery scores and features per day, in accordance with a representative embodiment.

In FIG. 6, Pearson correlation coefficient between the recovery scores and the created features per day are shown for the oncology training set. Vital signs are displayed as a first group, activity metrics as a second group, and combination features as a third group. The boundary used for feature selection may also be shown. FIG. 6 shows the Pearson correlation coefficient of the features with the engineered reference scores, which gives an indication of which parameters are likely to be linked to patient recovery for the oncology population. In the first set of embodiments, activity metrics have high correlation scores, but high correlation scores also exist for circadian rhythm in heart rate, some heart rate variability parameters in the frequency domain and certain vital sign/activity combination metrics such as heart rate recovery. Next to these features that are calculated per day, also change in feature values may also be used to add the history and the trend of the features to the trained model. These changes in feature values may be calculated by subtracting the feature value of the previous day from the value of the current day. Since no change in feature values can be calculated for the first day, no recovery prediction is made on that day.

A prediction model may be built using various machine learning algorithms as in FIG. 4, combined with various feature selection methods, and data imputation approaches. Different supervised machine learning algorithms may be tested using the method of FIG. 4, and these supervised machine learning algorithms include linear regression with Lasso, k-nearest neighbor, tree-based methods, such as decision tree, random forest and extreme gradient boosting, and support vector regression.

For the first set of embodiments, extreme gradient boosting method (XGBoost) may be selected as providing the best performance during the training phase. The extreme gradient boosting method may also be used to train a prediction model to predict scores for the test set.

However, many different machine learning approaches exist that may also succeed in predicting recovery scores. Furthermore, instead of machine learning, other approaches may be used. For example, rule-based schemes or analytic expressions may be used. Analytic expressions may be or include a function that has the input values as arguments, processes the input values according to some formulas, and returns the prediction score. Alternatively, instead of predicting scores, the features themselves may be directly used to cluster patients. For example, trends of heart rate recovery over time, or number of steps per day, etc. may be plotted and used to identify anomalous patterns by comparing to profiles of patient subgroups. Although a single parameter may not be as accurate in detecting, for example, discharge readiness, such a method may serve to identify specific parameters according to which a patient is recovering anomalously and that can be targeted with countermeasures. For example, if circadian rhythm is problematic, it may be investigated if sleep disturbances can be overcome, while a low activity level may be counteracted with physiotherapy or activity targets. Additionally, combinations of parameter profiles may be used. For example, to have a certain heart rate recovery may be within the expected confidence interval for normal recovery of the representative patient group, but not necessarily for the subgroup with a similar resting heart rate profile as that patient, such that the combination of parameters may still be used to flag the patient to a health care practitioner.

FIG. 7A illustrates predicted recovery versus expected recovery, in accordance with a representative embodiment.

In FIG. 7A, predicted recovery versus expected recovery is shown wherein each dot represents a day. Predicted recovery is the result of the trained model applied by the central computer 101 or the central computer 201. Expected recovery may be the baseline, median, or mean of recoveries for the group or subgroup used for comparison for the patient. Different patients may be shown by distinct colors, different symbols or different labels in visualizations such as FIG. 7A. The expected recovery reflects engineered reference recovery scores.

FIG. 7B illustrates recovery scores over time, in accordance with a representative embodiment.

in FIG. 7B, predicted recovery versus expected recovery scores are shown for two different patients over time. FIG. 7B shows that for the training set the trained model is able to predict recovery scores quite well, even though the resulting profiles are less smooth than the engineered reference profiles.

FIG. 8A, FIG. 8B and FIG. 8C illustrate individual predicted profiles compared to average profiles, in accordance with a representative embodiment.

In FIG. 8A, FIG. 8B and FIG. 8C, individual predicted profiles may be compared to the average profiles of the long length of stay (e.g., greater than eight days) and short length of stay (e.g., less than or equal to eight days) groups of the entire oncology dataset. The error bands are shown as the 10th and 90th percentile, and the events are visualized with vertical lines. On the test set the trained model may also be able to predict patient recovery scores in accordance with the expected profiles. The individual profiles may be compared to clusters of previous patient profiles, as shown in each of FIG. 8A, FIG. 8B and FIG. 8C. As an example, oncology patients may be grouped with a short length of stay as less than or equal to eight days and a long length of stay as greater than eight days. The patients may then be divided accordingly. While logically the profiles of the groups may partially overlap, such division can be used to identify individual patients likely to have a short length of stay as in FIG. 8A or long length of stay as in FIG. 8B and FIG. 8C. From the predicted recovery scores in FIG. 8A, it can be seen that already in the first days after surgery it is likely that the patient will have a short length of stay, which can help in discharge planning, for example by scheduling the patient for early discharge. According to the model a recovery score of 1.8 indicates discharge readiness. However, the recovery predictions should not replace current care, and other factors play a role that are not reflected in the recovery score. But the recovery score may serve as an objective metric in discharge readiness evaluations. Every day provides a new/refined prediction. Communication with the patient and health care providers should make clear that predictions are expectations, and not guarantees. FIG. 8B and FIG. 8C show patients with recovery profiles below the average of the long length of stay group. The patient in FIG. 8B was discharged on day nine, even though the predicted recovery score was still far below 1.8, and readmission occurred on day thirteen, indicating the original discharge may indeed have been too early. The patient in FIG. 8C also displays slow recovery compared to the reference profiles, and a decline three days in a row after day seven, that is followed by a complication on day eleven. A declining score for three days in a row towards the lower boundary of the long length of stay group may serve as trigger for health care providers to identify this patient as being in need of extra care, and potentially prevent the occurrence of a complication. Moreover, in the final model used for this example use case of predicting recovery for oncology patients, only six features are used to enhance interpretability, a combination of activity, circadian rhythm, heart rate recovery and delta features. By looking at the features it can be understood why the recovery scores are low, e.g. due to a small number of steps or upright hours, or poor circadian rhythm or heart rate recovery, which may point to different underlying issues and thereby help interpret patient condition.

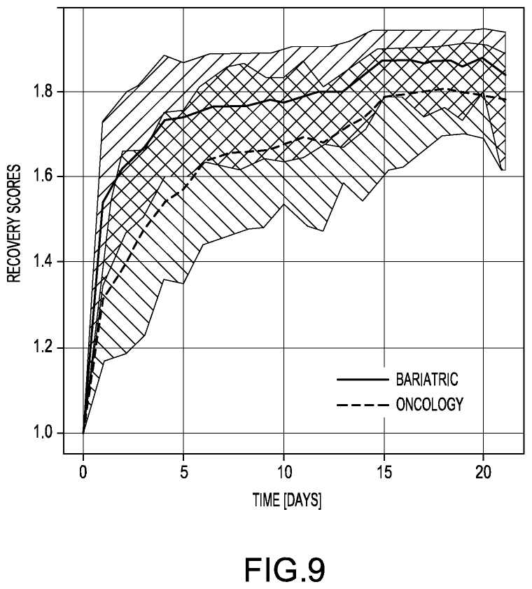

FIG. 9 illustrates predicted average recovery profiles, in accordance with a representative embodiment.

FIG. 9 illustrates predicted average recovery profiles of oncology and bariatric test set patient groups. The first set of example embodiments explained with respect to FIG. 5A, FIG. 5B, FIG. 5C, FIG. 5D, FIG. 5E, FIG. 5F, FIG. 6, FIG. 7A, FIG. 7B, FIG. 8A, FIG. 8B and FIG. 8C involves predictions for oncology patients after abdominal surgery. A similar approach may be used for different surgical patient groups, and a test of the model trained on the oncology group on a separate group of bariatric surgery patients shows that recoveries for this group may also be predicted over time. Indeed, the predicted recovery for the bariatric groups is faster compared to the oncology group, though this may be expected based on the shorter overall length of stay. The predicted recovery for the bariatric surgery group is shown in FIG. 9.

As shown in FIG. 9, the score of 1.8 which was defined as discharge ready is reached later than the actual discharge for most patients, indicating the trained model may need to be retrained for the bariatric patients. Additionally, or alternatively, different features or models may be used for the prediction for the bariatric surgery group. Furthermore, a different reference recovery score may be more appropriate to train a model for the bariatric patient group, as the patients may tend to have similar short lengths of stay and few complications and readmissions, which are the key differentiators for creating the reference profiles in the oncology patient group.

In the first set of embodiments, parameters related to vital signs, activity and combinations thereof are derived. However, for example, sleep metrics or metrics related to pain, psychological and social wellbeing may also be incorporated.

In the first set of embodiments, the features may be translated into a single value per day. However, different time windows may also be used. Furthermore, separate features may be combined using, for example, principal component analysis. Additionally, more extensive change of feature value features may be used. For example, features with multiple time points may be used, instead of only the change from the previous time point, as this may provide additional information on trends.

In some embodiments, raw data such as an accelerometer signal, ECG data, or PPG data may be taken to provide the recovery score. The physiological and ambulatory information important for estimating the recovery score may be extracted directly from the raw data, such as by using end-to-end deep learning.

In some embodiments, information of a previous recovery trend of the same patient may be used to increase the accuracy of the latest estimated recovery trend. This information may be useful for patients that are often readmitted in the hospital.

Grouping of patients described herein is not limited to two groups of recovery trends. Rather, in some embodiments an output at S355 may indicate the likelihood of a patient belonging to more than the two recovery trends of slow recovery or fast recovery. For example, the number of patient groups may be more than two. Additionally, after the patient is initially assigned to one recovery trend group, the recovery trend group may be further divided into subgroups in order to more accurately classify the patient. The splitting of the recovery trend groups may be performed iteratively until the trend groups reach a desired accuracy or specificity, or until the number of patients in each of the subgroups is no longer above a predetermined threshold.

The teachings herein are primarily directed to predicting recovery for individual patients. However, the trained models described herein may also be used to make predictions for entire hospitals or wards. For example, the recovery curve of an ensemble average over the patients in a hospital may be compared to the group of analogous curves from different hospitals. In this way, matters such infections in a hospital may be detected to reflect whether the hospital's curve is an outlier to the ensemble of other hospitals. Geographical density may also be determined in a comparable manner.

FIG. 10 illustrates a computer system, on which a method for anomalous patient recovery detection is implemented, in accordance with another representative embodiment.

Referring to FIG. 10, the computer system 1000 includes a set of software instructions that can be executed to cause the computer system 1000 to perform any of the methods or computer-based functions disclosed herein. The computer system 1000 may operate as a standalone device or may be connected, for example, using a network 1001, to other computer systems or peripheral devices. In embodiments, a computer system 1000 performs logical processing based on digital signals received via an analog-to-digital converter.

In a networked deployment, the computer system 1000 operates in the capacity of a server or as a client user computer in a server-client user network environment, or as a peer computer system in a peer-to-peer (or distributed) network environment. The computer system 1000 can also be implemented as or incorporated into various devices, such as a workstation that includes a controller, a stationary computer, a mobile computer, a personal computer (PC), a laptop computer, a tablet computer, or any other machine capable of executing a set of software instructions (sequential or otherwise) that specify actions to be taken by that machine. The computer system 1000 can be incorporated as or in a device that in turn is in an integrated system that includes additional devices. In an embodiment, the computer system 1000 can be implemented using electronic devices that provide voice, video or data communication. Further, while the computer system 1000 is illustrated in the singular, the term “system” shall also be taken to include any collection of systems or sub-systems that individually or jointly execute a set, or multiple sets, of software instructions to perform one or more computer functions.

As illustrated in FIG. 10, the computer system 1000 includes a processor 1010. The processor 1010 may be considered a representative example of a processor of a controller and executes instructions to implement some or all aspects of methods and processes described herein. The processor 1010 is tangible and non-transitory. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The processor 1010 is an article of manufacture and/or a machine component. The processor 1010 is configured to execute software instructions to perform functions as described in the various embodiments herein. The processor 1010 may be a general-purpose processor or may be part of an application specific integrated circuit (ASIC). The processor 1010 may also be a microprocessor, a microcomputer, a processor chip, a controller, a microcontroller, a digital signal processor (DSP), a state machine, or a programmable logic device. The processor 1010 may also be a logical circuit, including a programmable gate array (PGA), such as a field programmable gate array (FPGA), or another type of circuit that includes discrete gate and/or transistor logic. The processor 1010 may be a central processing unit (CPU), a graphics processing unit (GPU), or both. Additionally, any processor described herein may include multiple processors, parallel processors, or both. Multiple processors may be included in, or coupled to, a single device or multiple devices.

The term “processor” as used herein encompasses an electronic component able to execute a program or machine executable instruction. References to a computing device comprising “a processor” should be interpreted to include more than one processor or processing core, as in a multi-core processor. A processor may also refer to a collection of processors within a single computer system or distributed among multiple computer systems. The term computing device should also be interpreted to include a collection or network of computing devices each including a processor or processors. Programs have software instructions performed by one or multiple processors that may be within the same computing device or which may be distributed across multiple computing devices.

The computer system 1000 further includes a main memory 1020 and a static memory 1030, where memories in the computer system 1000 communicate with each other and the processor 1010 via a bus 1008. Either or both of the main memory 1020 and the static memory 1030 may be considered representative examples of a memory of a controller, and store instructions used to implement some or all aspects of methods and processes described herein. Memories described herein are tangible storage mediums for storing data and executable software instructions and are non-transitory during the time software instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The main memory 1020 and the static memory 1030 are articles of manufacture and/or machine components. The main memory 1020 and the static memory 1030 are computer-readable mediums from which data and executable software instructions can be read by a computer (e.g., the processor 1010). Each of the main memory 1020 and the static memory 1030 may be implemented as one or more of random access memory (RAM), read only memory (ROM), flash memory, electrically programmable read only memory (EPROM), electrically erasable programmable read-only memory (EEPROM), registers, a hard disk, a removable disk, tape, compact disk read only memory (CD-ROM), digital versatile disk (DVD), floppy disk, blu-ray disk, or any other form of storage medium known in the art. The memories may be volatile or non-volatile, secure and/or encrypted, unsecure and/or unencrypted.

“Memory” is an example of a computer-readable storage medium. Computer memory is any memory which is directly accessible to a processor. Examples of computer memory include, but are not limited to RAM memory, registers, and register files. References to “computer memory” or “memory” should be interpreted as possibly being multiple memories. The memory may for instance be multiple memories within the same computer system. The memory may also be multiple memories distributed amongst multiple computer systems or computing devices.

As shown, the computer system 1000 further includes a video display unit 1050, such as a liquid crystal display (LCD), an organic light emitting diode (OLED), a flat panel display, a solid-state display, or a cathode ray tube (CRT), for example. Additionally, the computer system 1000 includes an input device 1060, such as a keyboard/virtual keyboard or touch-sensitive input screen or speech input with speech recognition, and a cursor control device 1070, such as a mouse or touch-sensitive input screen or pad. The computer system 1000 also optionally includes a disk drive unit 1080, a signal generation device 1090, such as a speaker or remote control, and/or a network interface device 1040.

In an embodiment, as depicted in FIG. 10, the disk drive unit 1080 includes a computer-readable medium 1082 in which one or more sets of software instructions 1084 (software) are embedded. The sets of software instructions 1084 are an example of a computer product or a computer program product which, when executed, may be used to directly and/or indirectly implement one or more aspects of methods described herein, such as methods attributable to a controller. The set of software instructions 1084 are read from the computer-readable medium 1082 to be executed by the processor 1010. Further, the software instructions 1084, when executed by the processor 1010, perform one or more steps of the methods and processes as described herein. In an embodiment, the software instructions 1084 reside all or in part within the main memory 1020, the static memory 1030 and/or the processor 1010 during execution by the computer system 1000. Further, the computer-readable medium 1082 may include software instructions 1084 or receive and execute software instructions 1084 responsive to a propagated signal, so that a device connected to a network 1001 communicates voice, video or data over the network 1001. The software instructions 1084 may be transmitted or received over the network 1001 via the network interface device 1040.

In an embodiment, dedicated hardware implementations, such as application-specific integrated circuits (ASICs), field programmable gate arrays (FPGAs), programmable logic arrays and other hardware components, are constructed to implement one or more of the methods described herein. One or more embodiments described herein may implement functions using two or more specific interconnected hardware modules or devices with related control and data signals that can be communicated between and through the modules. Accordingly, the present disclosure encompasses software, firmware, and hardware implementations. Nothing in the present application should be interpreted as being implemented or implementable solely with software and not hardware such as a tangible non-transitory processor and/or memory.

In accordance with various embodiments of the present disclosure, the methods described herein may be implemented using a hardware computer system that executes software programs. Further, in an exemplary, non-limited embodiment, implementations can include distributed processing, component/object distributed processing, and parallel processing. Virtual computer system processing may implement one or more of the methods or functionalities as described herein, and a processor described herein may be used to support a virtual processing environment.

Accordingly, anomalous patient recovery detection enables improved accuracy in patient care planning, physical therapy planning and improved resource utilization.

Although anomalous patient recovery detection has been described with reference to several exemplary embodiments, it is understood that the words that have been used are words of description and illustration, rather than words of limitation. Changes may be made within the purview of the appended claims, as presently stated and as amended, without departing from the scope and spirit of anomalous patient recovery detection in its aspects. Although anomalous patient recovery detection has been described with reference to particular means, materials and embodiments, anomalous patient recovery detection is not intended to be limited to the particulars disclosed. Rather, anomalous patient recovery detection extends to all functionally equivalent structures, methods, and uses such as are within the scope of the appended claims.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

1. A system, comprising:
a memory that stores instructions; and
a processor that executes the instructions, wherein, when executed by the processor, the instructions cause the system to:
repeatedly obtain a plurality of types of data derived from a recovering patient;
apply the plurality of types of data to a trained model;
repeatedly generate a recovery score predicting recovery of the recovering patient; and
output the recovery score.

2. The system of claim 1, wherein, when executed by the processor, the instructions further cause the system to:
establish a baseline;
compare the recovery score to the baseline.

3. The system of claim 1, wherein, when executed by the processor, the instructions further cause the system to:
generate an alarm based on comparing the recovery score to a baseline.

4. The system of either of claim 1, wherein, when executed by the processor, the instructions further cause the system to:
generate an alarm based on a trend of recovery scores including the recovery score.

5. The system of claim 1, wherein the system is centralized and generates and outputs recovery scores for a plurality of recovering patients.

6. The system of claim 1, wherein the system generates and outputs recovery scores for recovering patients of a plurality of medical facilities.

7. The system of claim 1, wherein the trained model comprises an extreme gradient boosting-based model.

8. The system of claim 1,
wherein the plurality of types of data includes data that are derived from vital signs of the recovering patient.

9. The system of claim 1,
wherein the plurality of types of data includes data that are derived from activity metrics for the recovering patient.

10. The system of claim 1,
wherein the plurality of types of data further includes data that are derived from vital signs of the recovering patient.

11. A method performed by a central system, comprising:
repeatedly obtaining a plurality of types of data derived from a recovering patient;
applying, by a processor that executes instructions from a memory, the plurality of types of data to a trained model;
repeatedly generating a recovery score predicting recovery of the recovering patient; and
outputting the recovery score.

12. The method of claim 11, further comprising:
establishing a baseline; and
comparing the recovery score to the baseline.

13. The method of claim 11, further comprising:
generating an alarm based on comparing the recovery score to a baseline.

14. The method of claim 11, further comprising:
generating an alarm based on a trend of recovery scores including the recovery score.

15. The method of claim 11, wherein the central system generates and outputs recovery scores for a plurality of recovering patients.

16. The method of claim 11, wherein the central system generates and outputs recovery scores for recovering patients of a plurality of medical facilities.

17. The method of claim 11, wherein the trained model comprises an extreme gradient boosting-based model.

18. The method of claim 11,
wherein the plurality of types of data are derived from vital signs of the recovering patient.

19. The method of claim 11,
wherein the plurality of types of data are derived from activity metrics for the recovering patient.

20. The method of claim 11,
wherein the plurality of types of data further includes data that are derived from vital signs of the recovering patient.

21. A tangible non-transitory computer readable storage medium that stores a computer program, wherein the computer program, when executed by a processor, causes a system to:
repeatedly obtain a plurality of types of data derived from a recovering patient;
apply the plurality of types of data to a trained model;
repeatedly generate a recovery score predicting recovery of the recovering patient; and
output the recovery score.
